(19) United States
(12) Patent Application Publication
(10) Pub. No.: US 2021/0228443 A1
(43) Pub. Date: Jul. 29, 2021

(54) RECONSTITUTION AND MIXING SYSTEMS

(71) Applicant: ADVANCED MEDICAL SOLUTIONS ISRAEL (SEALANTIS) LTD, Haifa (IL)

(72) Inventor: Ohad KIMHI, Kiryat Yam (IL)

(21) Appl. No.: 17/052,924

(22) PCT Filed: May 5, 2019
(86) PCT No.: PCT/IL2019/050500

§ 371 Date: Nov. 4, 2020

Related U.S. Application Data
(60) Provisional application No. 62/666,776, filed on May 4, 2018.

(51) Int. Cl. A61J 1/20; A61L 24/04; A61L 24/08; A61L 24/10; B01F 1/00

(52) U.S. Cl. CPC A61J 1/2048; A61L 24/04; A61L 24/08; B01F 2215/0032; B01F 1/0027; B01F 2215/0034; A61L 24/10

(57) ABSTRACT

An adapter including at least one wall defining a chamber, at least one opening within the wall, and a water-soluble additive coating at least a portion of the wall such that fluid flowing from one opening through the chamber dissolves the water-soluble additive within the chamber.

### CROSS REFERENCE TO RELATED APPLICATIONS

This application claims the benefit of priority to U.S. Provisional patent Application No. 62/666,776 filed May 4, 2018, entitled “RECONSTITUTION AND MIXING SYSTEMS” the contents of which are incorporated herein by reference in their entirety.

### FIELD OF THE INVENTION

The present invention, in some embodiments thereof, relates to an adapter and, more particularly, but not exclusively, to an adapter comprising additive coating.

### BACKGROUND

Many pharmaceuticals and other medical-related products such as tissue adhesives are composed of several ingredients that must be kept separately before application. One example is a lyophilized drug that needs to be mixed with diluent shortly before injection. Another example is a tissue adhesive that should be mixed with its curing agent just before applying it. Other examples are mixing of two or more ingredients that might react with each other or degrade one another. It is a common practice is to pack these products in separate prefilled containers such as syringes or vials. To allow application, all containers are connected to each other using adapters, which may have an additional mixing tip connected to it.

Commonly used adapters connect between two pieces of equipment, for example, a syringe and dispensing equipment. Such adapters are commonly used for a manual, more controlled dispense of fluid.

### SUMMARY OF THE INVENTION

The following embodiments and aspects thereof are described and illustrated in conjunction with systems, tools and methods which are meant to be exemplary and illustrative, not limiting in scope.

According to an aspect of some embodiments of the invention there is provided an adapter, comprising: at least one wall defining a chamber; at least one opening within the wall; a water-soluble additive coating at least a portion of the wall such that fluid flowing from one opening through the chamber dissolves the additive within the chamber.

In some embodiments, the water-soluble additive comprises an adhesiveness property in relation to the wall.

In some embodiments, the water-soluble additive is inert to the fluid.

In some embodiments, the water-soluble additive dissolves homogenously within the fluid.

In some embodiments, the water-soluble additive is selected from the group consisting of: a dye, a pharmaceutical, and a pharmaceutical excipient or any combination thereof.

In some embodiments, the water-soluble additive is a dye.

In some embodiments, the coating further comprises a water-soluble adhesive.

In some embodiments, the coating comprises the water-soluble additive and adhesive at a w/w (weight/weight) ratio of 1:1 to 1:50.

In some embodiments, the adhesive is selected from the group consisting of: a water-soluble saccharide, a peptide, a protein, a low-molecular weight surfactant, and a water-soluble adhesive polymer.

In some embodiments, the water-soluble adhesive polymer is selected from: a poly-phosphazene, a poly-phosphoester, a poly-oxazoline, a poly-[divinylether-maleic anhydride], a poly[N(2-hydroxypropyl)methacrylamide], a poly-acrylamide, a poly-acrylic acid, a poly-vinyl alcohol, a poly-vinyl pyrrolidone, a poly-ethylene glycol or any combination thereof.

In some embodiments, the water-soluble saccharide is selected from a monosaccharide, an oligosaccharide, and a polysaccharide or any combination thereof.

In some embodiments, the water-soluble adhesive is a monosaccharide.

In some embodiments, the opening is configured to couple to at least one of a vial, a syringe, a compartment, a pipe, a funnel, a beaker, a tube, a pipet, and an adapter.

In some embodiments, the chamber comprises at least two openings and at least one channel between the opening.

In some embodiments, at least a portion of at least one channel is coated by the water-soluble additive.

In some embodiments, the chamber comprises at least two openings and the path of the channel between the openings is longer than the minimal distance between the openings.

In some embodiments, at least a portion of the surface of the wall within the chamber is irregular, textured, and/or comprises irregularities.

In some embodiments, at least a portion of the surface of the channel is irregular, textured, and/or comprises irregularities.

In some embodiments, the irregular surface, textured surface, and/or irregularities comprise at least one of a nanostructure, microstructure, or any combination thereof.

In some embodiments, the water-soluble additive coats at least a portion of the irregular surface, textured surface, and/or irregularities.

In some embodiments, the surface area of at least a portion of the inner surface of the wall inner surface is larger than the surface area of the corresponding outer surface of the portion of the wall in relation to the chamber.

According to an aspect of some embodiments of the invention there is provided a kit for mixing an additive with fluid, comprising: at least one of fluid source and/or a fluid drain; and at least one adapter configured to couple to the fluid source and/or the fluid drain, comprising at least one wall defining a chamber, at least one opening within the wall configured to couple to the fluid source and/or fluid drain; and a water-soluble additive coating at least a portion of the wall such that fluid flowing within the chamber dissolves the additive.

In some embodiments, the fluid source and/or fluid drain is at least one of a vial, a syringe, a compartment, a pipe, a funnel, a beaker, a tube, a pipet, and an adapter.

According to an aspect of some embodiments of the invention there is provided a method for mixing water-soluble additive with fluid, comprising streaming fluid into an adapter through at least one opening; streaming fluid through a chamber of the adapter such that water-soluble additive coating at least a portion of the wall of the chamber dissolves the fluid; and ejecting a homogenous mixture of the fluid and the water-soluble additive through at least one opening of the adapter.

According to an aspect of some embodiments of the invention there is provided a method of coating an adapter, comprising mixing a water-soluble additive with a solvent, thereby obtaining a coating solution; providing the coating solution, to the adapter; drying the coating solution at 10 to 90° C., thereby coating the adapter.

In some embodiments, the method further comprises mixing an adhesive and the additive.

In some embodiments, a w/w (weight/weight) ratio of the adhesive to the additive ranges from 1:1 to 1:50 within the coating solution.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 is an adapter, in accordance with some embodiments of the present invention;

FIGS. 2A, 2B, 2C and 2D are adapters for mixing an additive with fluid, in accordance with some embodiments of the present invention;

FIG. 3 is a kit for mixing an additive with fluid, in accordance with some embodiments of the present invention; and

FIG. 4 is a method for mixing an additive with fluid, in accordance with some embodiments of the present invention.

### DETAILED DESCRIPTION OF THE INVENTION

According to an aspect of some embodiments of the present invention, there is provided an adapter configured to couple with at least one of a fluid source and/or a fluid drain, comprising an inner coat comprising an additive such that fluid entering the adapter is mixed, reconstituted, or dissolved in/with the additive. In some embodiments, the additive is a water-soluble additive.

According to an aspect of some embodiments of the present invention, there is provided an adapter comprising an inner coat, wherein the coat comprises an adhesive and an additive, wherein the adhesive is bound to a surface of the adapter and the additive is incorporated within the adhesive, wherein the adhesive and the additive are soluble in an aqueous solution, and wherein the w/w (weight/weight) ratio of the additive to the adhesive in the coating ranges from 1:1 to 1:50.

According to an aspect of some embodiments of the present invention, there is provided a kit for mixing an additive with fluid, comprising an adapter and at least one of a fluid source and/or fluid drain. In some embodiments, the adapter comprises a chamber and at least one opening within the chamber wall, configured to couple to one or more fluid drain and/or fluid source. In some embodiments, at least a portion of the chamber wall is coated with an additive such that that fluid flowing between the fluid source and the fluid drain through the chamber interacts with the additive.

According to an aspect of some embodiments of the present invention, there is provided a method for mixing an additive with fluid. In some embodiments, the method comprises streaming fluid into an adapter through one or more opening. In some embodiments, the method comprises streaming fluid through a chamber of the adapter such that an additive coating at least a portion of the wall of the chamber mixes, reconstitutes and/or dissolves with the fluid. In some embodiments, the method comprises ejecting the homogenous mixture of the fluid and the additive through at least one opening of the adapter.

According to an aspect of some embodiments of the present invention, there is provided a method of coating an adapter. In some embodiments, the method comprises mixing an additive and an adhesive (carrier/encapsulant) at a w/w (weight/weight) ratio ranging from 1:1 to 1:50 with an aqueous buffer, thereby obtaining an aqueous solution. In some embodiments, the method comprises providing the aqueous solution to the adapter. In some embodiments, the method comprises drying the aqueous solution at 10 to 90° C., thereby obtaining the coated adapter.

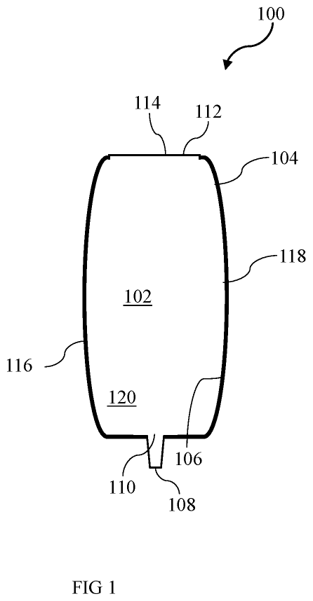

Reference is made to FIG. 1, which is an adapter, in accordance with some embodiments, of the present invention. In some embodiments, the adapter 100 comprises at least one wall 104 defining a chamber 102. In some embodiments, the adapter 100 comprises at least one opening 110/114 within the wall 104. In some embodiments, the adapter 100 comprises an additive 118 coating at least a portion of the wall 104 such that that fluid flowing from one opening 110/114 through the chamber 102 interacts with the additive 118. In some embodiments, the adapter 100 comprises an additive 118 coating at least a portion of the wall 104 such that that fluid flowing from one opening 110/114 to another opening 110/114 through the chamber 102 interacts with the additive 118.

In some embodiments, the wall 104 comprises an inner surface 106 and an outer surface 116. In some embodiments, the additive 118 coats the inner surface 106. In some embodiments, the additive 118 is adhered to at least a portion of the wall 104 and/or a portion of the inner surface 106. In some embodiments, the additive 118 is adhered by an adhesive.

A potential advantage of the additive 118 coating at least a portion of the inner surface 106 is in that fluid flowing through the chamber 102 contacts, dissolves and/or interacts with the additive 118. In some embodiments, the additive 118 is one or more of a dye, ingredient in a drug, and/or an ingredient for an adhesive, which coats the wall 104 inner surface 106 of a chamber 102 within the adapter 100. In some embodiments, the additive 118 is a solid or semi-solid.

In some embodiments, the chamber 102 comprises a hollow portion, such as a tube or hollow cylinder. In some embodiments, the volume of the chamber 102 is 10-80 mL. In some embodiments, the volume of the chamber 102 is 20-60 mL. In some embodiments, the chamber 102 wall 104 is rigid, semi-rigid, and/or flexible.

In some embodiments, the wall 104 comprises one or more opening 110/114. In some embodiments, the wall 104 comprises one or more openings 110/114 positioned on opposing sides of the chamber 102. In some embodiments, and as described in greater detail elsewhere herein, the opening 110/114 is configured to couple to one or more of a fluid source and/or fluid drain. In some embodiments, the opening 110/114 comprises a coupling mechanism couplable to one or more of a fluid source and/or fluid drain.

In some embodiments, and as described in greater detail elsewhere herein, one or more opening 110/114 of the adapter 100 is coupled to one or more fluid source and/or fluid drain through a connector, e.g., a pipe.

In some embodiments, the coupling mechanism is one or more of a needle 108 and a luer lock 112. For example, a needle 108 coupled to the adapter 100 penetrates a fluid source such that the chamber 102 is in fluid communication with the fluid source through the needle 108.

In some embodiments, the coupling mechanism between an opening 110/114 and one or more of the fluid source and the fluid drain comprises a lock and key configuration (e.g., an opening 110/104 comprises a threaded portion which is compatible with a raised portion of the fluid source and/or fluid drain), a screw-on mechanism, a pressure lock, a sealed lock, or any combination thereof.

In some embodiments, more than one opening 110/114 is couplable to more than one fluid source. In some embodiments, more than one opening 110/114 is couplable to more than one fluid drain.

A potential advantage of the adapter 100 comprising more than one opening 110/114 couplable to more than one fluid source is in that a plurality of ingredients can be kept separate before entering the chamber 102.

In some embodiments, the chamber 102 comprise one or more channels 120 between a plurality of openings 110/114. In some embodiments, at least a portion of the channel wall 218 is coated by the additive 118. In some embodiments, an adhesive adheres between the channel wall 218 and the additive 118. In some embodiments, at least a portion of the inner surface 106 defines channel 120.

A potential advantage of the chamber 102 comprising at least one channel 120 is in that the fluid flowing within the chamber 102 interacts with the surfaces of the channel wall 218 and/or the inner surface 106 chamber 102 thereby having more interaction with the additive-coated surfaces than a chamber 102 without any channels 120.

In some embodiments, the surface area of at least a portion of the wall 104 inner surface 106 is larger than the surface area of the corresponding outer surface of the portion of the wall 104. In some embodiments, at least a portion of the inner surface 106 and/or channel 120 is irregular, textured, and/or comprises irregularities. In some embodiments, the irregular surface, textured surface, and/or irregularities comprise one or more of a nanostructure, microstructure, or any combination thereof. In some embodiments, the additive 118 coats at least a portion of the irregular surface, textured surface, and/or irregularities.

A potential advantage of at least a portion of the inner surface 106 and/or channel 120 comprising an irregular surface, textured surface, and/or irregularities at least partially coated by an additive 118 is in that fluid flowing through the chamber 102 interacts with a larger surface area of the coat of additive 118 than a fluid flowing through a chamber comprising no irregular surface, textured surface, and/or irregularities coated by an additive 118.

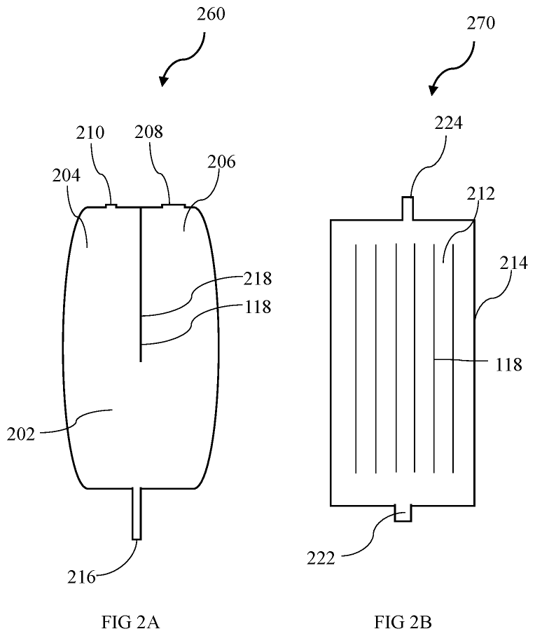

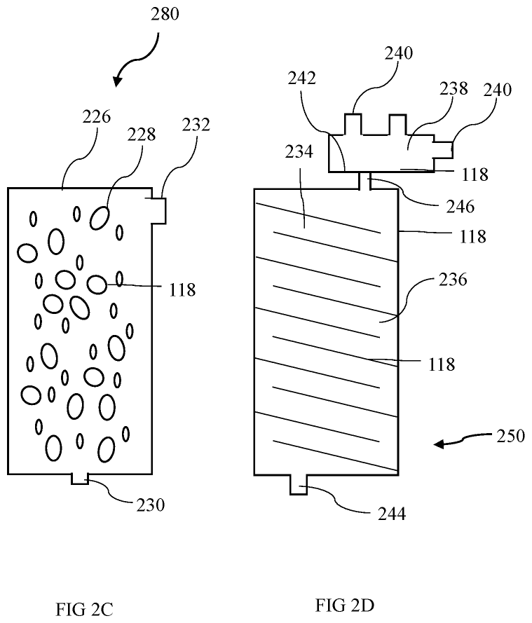

In some embodiments, such as depicted by FIGS. 2A, 2B, 2C and 2D, which are adapters for mixing an additive with fluid, in accordance with some embodiments of the present invention, the one or more channels 120 guide the flow of fluid within the chamber 102. In some embodiments, the one or more channels 120 guide the flow of fluid from one opening 110/114 to another opening 110/114 and/or back into the same opening 110/114. In some embodiments, such as depicted by FIG. 2A, the channel 202 splits into a plurality of separate channels 204/206, each being configured to direct fluid from an opening 216 through a plurality of separate openings 210/208, respectively. In some embodiments, fluid is streamed into the chamber 202 through one or more openings 210/208 and streamed out of the chamber 202 through an additional opening, e.g., opening 216. In some embodiments, such as depicted by FIG. 2B, the chamber 214 comprises a plurality of channels 214 configured to direct a fluid from one opening 224 to another opening 222 and/or from one opening 224 to the same opening 224.

In some embodiments, such as depicted by FIG. 2C, the chamber 226 is a solid comprising a plurality of pores 228, coated by an additive 118, through which the one or more opening 232/230 is in fluid communication with the pores 228. In some embodiments, such as depicted by FIG. 2D, the path of the channel 234 is longer than the minimal distance between the one or more openings 244/246 through which the fluid is streamed.

In some embodiments, such as depicted by FIG. 2D, the adapter 100/250/260/270/280 is coupled to a mixing tip 238. In some embodiments, and as described in greater detail elsewhere herein, the mixing tip 238 is coupled between a plurality of fluid sources 240 and the adapter 100/250/260/270/280. In some embodiments, a mixture flows from the mixing tip 238 into the adapter 100/250/260/270/280. In some embodiments, at least a portion of the inner surface 242 of the mixing tip 238 is coat by the additive 118.

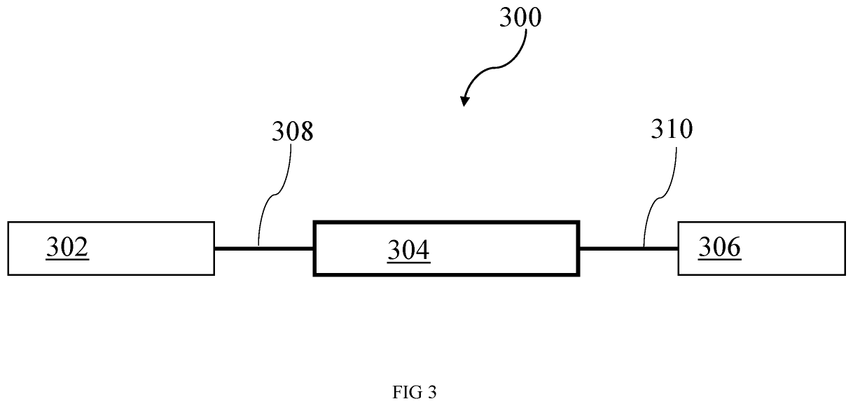

Reference is made to FIG. 3, which is a kit for mixing an additive with fluid, in accordance with some embodiments of the present invention. In some embodiments, the kit 300 comprises at least one adapter 100/250/260/270/280/304 configured to couple with at least one or more of a fluid source 306 and/or a fluid drain 302. In some embodiments, the kit 300 comprises at least one of a fluid source 306 and/or a fluid drain 302. In some embodiments, the fluid source 306 is one or more of a vial, a syringe, a compartment, a pipe, a funnel, a beaker, a tube, a pipet, and an adapter. In some embodiments, the fluid drain 302 is one or more of a vial, a syringe, a compartment, a pipe, a funnel, a beaker, a tube, a pipet, and an adapter.

For example, in some embodiments, the adapter 100 is used for reconstitution, e.g., by mixing the content of a vial or a syringe with a diluent.

In some embodiments, the fluid source 306 supplies fluid to and/or from the adapter 100/250/260/270/280/304. In some embodiments, the fluid drain 302 supplies fluid to and/or from the adapter 100/250/260/270/280/304.

In some embodiments, the kit 300 comprises a mixing tip 238. In some embodiments, the adapter 100/250/260/270/280/304 and/or mixing tip 238 is smaller than the fluid source and/or fluid drain. In some embodiments, the mixing tip 238 and/or the adapter 100/250/260/270/280/304 is coated with the additive 118 and/or an additional ingredient.

A potential advantage of the mixing tip 238 and/or the adapter 100/250/260/270/280/304 comprising the additive 118 and/or an additional ingredient is in that specific amounts of the additive 118 and/or an additional ingredient are used to mix with a fluid flowing from the fluid source 306 and/or fluid drain 302, which enables higher accuracy than manually adding the additive 118 and/or an additional ingredient.

In some embodiments, the kit 300 comprises a fluid reservoir which supplies fluid for one or more of the fluid drain 302 and/or the fluid source 306.

In some embodiments, the kit 300 comprises one or more connector 308/310 configured to couple between the adapter 100/250/260/270/280/304 and one or more of fluid source 306 and/or fluid drain 302. In some embodiments, the connector 308/310 is a pipe, tube, channel, or the like. In some embodiments, the adapter 100/250/260/270/280/304 is in fluid communication with one or more of the fluid source 306 and/or the fluid drain 302 through the connector 308/310.

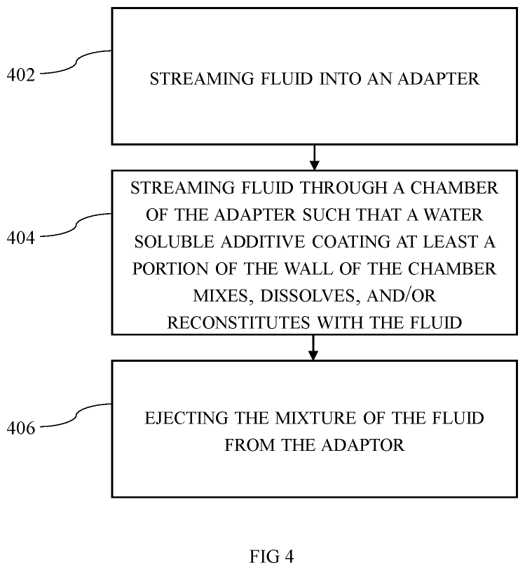

Reference is made to FIG. 4, which is a method for mixing an additive with fluid in accordance with some embodiments of the present invention. In some embodiments, at step 402, the method comprises streaming fluid into an adapter 100/250/260/270/280/304 through one or more opening. In some embodiments, at step 404, the method comprises streaming fluid through a chamber of the adapter such that an additive coating at least a portion of the wall of the chamber mixes, dissolves, and/or reconstitutes with the fluid. In some embodiments, at step 406, the method comprises ejecting a homogenous mixture of the fluid and the additive through a second opening of the adapter.

For example, in some embodiments, the adapter 100/250/260/270/280/304 comprises the additive 118, e.g., a dried drug or dye. In some embodiments, the fluid source 306 is a vial containing a fluid, e.g., a fluid diluent and is coupled to the adapter 100/250/260/270/280/304. In some embodiments, the fluid drain 302 is a syringe and is coupled to the adapter 100/250/304. In some embodiments, the fluid drain 302 is used to remove fluid pressure from the adapter 100/250/260/270/280/304.

In some embodiments, removing fluid pressure from the adapter 100/250/260/270/280/304 ejects the fluid from the fluid source 306 and into the adapter 100/250/260/270/280/304 (step 402). In some embodiments, the fluid streaming into the adapter 100/250/260/270/280/304 is mixed with the additive 118, e.g., a dried drug (step 404). In some embodiments, the additive 118 is reconstituted and/or dissolved within the fluid. In some embodiments, the fluid drain 302 applies negative pressure to the adapter 100/250/260/270/280/304 such that the mixture of fluid and additive 118, e.g., reconstituted drug and/or dyed fluid, flows from the adapter 100/250/260/270/280/304 into the fluid drain 302.

In some embodiments, the fluid source 306 comprises a dried diluent. In some embodiments, a fluid is streamed into the fluid source 306 through the adapter 100/250/260/270/280/304, which reconstitutes the dried diluent into a fluid diluent. In some embodiments, the fluid for reconstituting the dried diluent is ejected from the fluid drain 302 into the adapter 100/250/260/270/280/304.

### Coating

In some embodiments, the coating comprises a water-soluble additive.

The term “water-soluble”, as used herein means sufficiently soluble in an aqueous solution, such as water or a solution based primarily on water (e.g. a buffer, or a salt solution).

In some embodiments, a water-soluble additive has adhesive properties to the adapter. In some embodiments, a water-soluble additive has adhesive properties to a wall of the adapter. In some embodiments, a water-soluble additive is bound to the wall. In some embodiments, a water-soluble additive forms non-covalent bonds with the wall. In some embodiments, a water-soluble additive forms van der Waals bonds with the wall. In some embodiments, a water-soluble additive forms p-p stacking interactions with the wall.

The term “non-covalent bonds” encompasses ligand-receptor interactions, hydrogen bonding, dipole-dipole interactions and van der Waals bonds or any combination thereof. In terms of the present invention, non-covalent bond formation between the adhesive and the surface of the adapter provides adhesive properties to the coating.

In some embodiments, the coating further comprises a water-soluble adhesive which is bound to the wall. In some embodiments, a water-soluble adhesive forms non-covalent bonds with the wall. In some embodiments, a water-soluble adhesive forms hydrogen bonds with the wall.

In some embodiments, the coating comprises a water-soluble adhesive and a water-soluble additive. In some embodiments, the coating comprises an adhesive which encapsulates an additive. In some embodiments, an additive is absorbed to an adhesive. In some embodiments, an additive is dispersed in an adhesive. In some embodiments, an adhesive and an additive are bound via non-covalent bonds. In some embodiments, an adhesive and an additive are bound via hydrogen bonds. In some embodiments, an adhesive and an additive are bound via electrostatic bonds. In some embodiments, an adhesive and an additive are bound via van der Waals bonds.

In some embodiments, the coating is in a solid form. In some embodiments, the coating is a dry solid. In some embodiments, the dry solid consists less than 10% water. In some embodiments, the water content of the dry solid is less than 5% w/w. In some embodiments, the water content of the dry solid is less than 2% w/w. In some embodiments, the water content of the dry solid is less than 1% w/w.

In some embodiments, the coating forms a film on a wall of the adapter. In some embodiments, the coating forms a stable layer on the wall of the adapter. In some embodiments, the coating is bound to the wall.

In some embodiments, the coating covers the wall of the adapter. In some embodiments, the coating is a partial coating, covering at least a part of the wall.

In some embodiments, the coating forms a uniform layer on the wall. In some embodiments, the coating forms a layer with differential thickness. In either embodiment, the thickness of a coating layer is not limited.

In some embodiments, the w/w ratio of an additive to an adhesive in the coating ranges from 1:1 to 1:50. In some embodiments, the w/w ratio of an additive to an adhesive in the coating ranges from 1:1 to 1:3. In some embodiments, the w/w ratio of an additive to an adhesive in the coating ranges from 1:3 to 1:5. In some embodiments, the w/w ratio of an additive to an adhesive in the coating ranges from 1:5 to 1:8. In some embodiments, the w/w ratio of an additive to an adhesive in the coating ranges from 1:8 to 1:10. In some embodiments, the w/w ratio of an additive to an adhesive in the coating ranges from 1:10 to 1:15. In some embodiments, the w/w ratio of an additive to an adhesive in the coating ranges from 1:15 to 1:20. In some embodiments, the w/w ratio of an additive to an adhesive in the coating ranges from 1:20 to 1:30. In some embodiments, the w/w ratio of an additive to an adhesive in the coating ranges from 1:30 to 1:40. In some embodiments, the w/w ratio of an additive to an adhesive in the coating ranges from 1:40 to 1:50.

In some embodiments, the coating is soluble in the fluid. In some embodiments, the coating dissolves homogenously within the fluid. In some embodiments, after contacting the coating with the fluid, the coating releases an additive. In some embodiments, after dissolving the coating, a solution comprising an additive is formed. In some embodiments, the solution is a homogenous additive solution. In some embodiments, the solution is a homogenous solution comprising an adhesive and an additive is formed. In some embodiments, the solution is an aqueous solution.

In some embodiments, the coating does not undergo a chemical reaction upon contacting with the fluid. In some embodiments, the coating does not undergo a chemical reaction upon dissolution. In some embodiments, the coating does not undergo a chemical reaction with an aqueous solution. In some embodiments, the coating does not undergo a chemical reaction with the compartment of the vial. In some embodiments, a solution comprising the coating does not undergo a chemical reaction with the compartment of the vial. In some embodiments, an aqueous solution comprising the coating does not undergo a chemical reaction with the compartment of the vial. In some embodiments, the coating does not have a therapeutic activity. In some embodiments, the coating does not affect a therapeutic activity of an active ingredient.

In some embodiments, the coating further comprises an additional ingredient selected from the group consisting of: a tackifier, a crosslinking agent, and a binder or any combination thereof.

A tackifier may be used to increase a stickiness of the surface of the adhesive. Such agents may be useful for combining the compound of the invention with carriers to yield a coating composition. Such compositions may aid to maintain contact between the compound of the invention or a composition containing thereof, and a weed.

Non-limiting examples of adherents include but are not limited to: a lecithin, formononetin, alkali formononetinate, hesperetin, polyvinyl acetate, a cephalin, Mineral Oil, Arabino-galactan, Glycerol, Triethylene glycol, Vinyl Acetate, Polystyrene, and Polyvinyl.

Non-limiting examples of crosslinking agents include but are not limited to: an isocyanate compound, a melamine compound, a poly(meth)acrylate compound, an epoxy compound, and the like.

Binders may be used to impart cohesiveness of the coating.

Non-limiting examples of binders include but are not limited to: hydroxy propyl methyl cellulose, hydroxy propyl cellulose, hydroxy ethyl cellulose, ethyl cellulose, cellulose derivatives, maize starch, polyvinylpyrrolidone 25 alone or in combination with polyethylene glycols and the like. Binders may be used in the range of about 5% to about 30% by weight of total composition.

In some embodiments, the coating is a multilayer coating. In some embodiments, the multilayer coating comprises a first layer bound to the wall of the adapter, and a second layer. In dome embodiments, the second layer is a top layer. In some embodiments, the second layer is bound to the first layer. In some embodiments, the second layer is devoid of an additive. In some embodiments, the second layer is a protective layer. In some embodiments, the second layer prevents the first layer from degradation, such as abrasion or bleaching. In some embodiments, the second layer comprises an adhesive, as described hereinbelow. In some embodiments, the second layer further comprises an additional ingredient selected from the group consisting of: a tackifier, a crosslinking agent, and a binder or any combination thereof.

### Adhesive

In some embodiments, a water-soluble adhesive is selected from the group consisting of: a water-soluble saccharide, a peptide, a protein, a low-molecular weight surfactant, and a water-soluble adhesive polymer or any combination thereof.

In some embodiments, the adhesive is soluble in the fluid. In some embodiments, the adhesive dissolves homogenously within the fluid.

In some embodiments, the adhesive does not undergo a chemical reaction upon contacting with the fluid. In some embodiments, the adhesive does not undergo a chemical reaction upon dissolution. In some embodiments, the adhesive does not undergo a chemical reaction with an aqueous solution. In some embodiments, the adhesive does not undergo a chemical reaction with the compartment of the vial. In some embodiments, a solution comprising the adhesive does not undergo a chemical reaction with the compartment of the vial. In some embodiments, an aqueous solution comprising the adhesive does not undergo a chemical reaction with the compartment of the vial. In some embodiments, the adhesive does not have a therapeutic activity. In some embodiments, the adhesive does not affect a therapeutic activity of an active ingredient.

In some embodiments, the adhesive has adhesiveness to the surface of the adapter. In some embodiments, the adhesive facilitates bonding of the additive to the surface of the adapter. In some embodiments, the adhesive facilitates adhering of the additive to the surface of the adapter. In some embodiments, the adhesive forms non-covalent bonds with the wall of the adapter and with an additive. In some embodiments, the adhesive has at least one functional group selected from: an aromatic ring, an alkyl chain, a charged residue, a hydrogen bond donor or acceptor. In some embodiments, the adhesive is an amphiphilic substance containing a hydrophobic segment and a hydrophilic segment.

In some embodiments, the adhesive is a water-soluble adhesive polymer.

The term “adhesive polymer” used herein means a polymer, which exhibits adhesion to a surface.

Non-limiting examples of water soluble adhesive polymers include but are not limited to: poly-phosphazene, poly-phosphoester, poly-oxazoline, a poly-[divinylether-maleic anhydride], poly[N(2-hydroxypropyl)methacrylamide], poly-acrylamide, poly-acrylic acid, poly-vinyl alcohol, poly-vinyl pyrrolidone, poly-ethylene glycol or any combination thereof.

Non-limiting examples of low-molecular weight surfactants include but are not limited to: Polysorbate 80, sodium dodecyl sulfate, sunflower lecithin, egg lecithin, egg phosphatidylglycerol, soy lecithin, hydrogenated soy lecithin, and sphingomyelin.

In some embodiments, the adhesive is a saccharide.

As used herein, the term “saccharide” is referred to any water-soluble, optionally substituted mono-, di-, oligo-, or polysaccharide.

Non-limiting examples of water-soluble saccharides include but are not limited to: monosaccharide (e.g. fructose, glucose, galactose), a disaccharide (e.g. sucrose), an oligosaccharide (e.g. raffinose, maltotriose), a polysaccharide (e.g. cellulose acetate, gum arabic, gum ghatti, dextran, pullulan, amylopectin), and a substituted sugar (e.g. aminoglycoside).

In some embodiments, the adhesive is a monosaccharide. In some embodiments, the adhesive is a disaccharide. In some embodiments, the adhesive is an oligosaccharide.

### Additive

In some embodiments, a water-soluble additive is selected from a dye, a pharmaceutical, and a pharmaceutical excipient or any combination thereof.

In some embodiments, the additive is soluble in the fluid. In some embodiments, the additive dissolves homogenously within the fluid. In some embodiments, the additive dissolves homogenously in an aqueous solution.

In some embodiments, the additive does not undergo a chemical reaction upon contacting with the fluid. In some embodiments, the additive does not undergo a chemical reaction upon dissolution. In some embodiments, the additive does not undergo a chemical reaction with an aqueous solution. In some embodiments, the additive does not undergo a chemical reaction with the compartment of the vial. In some embodiments, a solution comprising the additive does not undergo a chemical reaction with the compartment of the vial. In some embodiments, an aqueous solution comprising the additive does not undergo a chemical reaction with the compartment of the vial. In some embodiments, the additive does not have a therapeutic activity. In some embodiments, the additive does not affect a therapeutic activity of an active ingredient.

Non-limiting examples of dyes include but are not limited to: anionic dyes (e.g. Alizarine Pure Blue B, Acid red 88), cationic dyes (e.g. methine dyes, anthraquinone dyes, azo dyes), and neutral dyes (e.g. Neutral Orange RL, Neutral Red GRL, Neutral Gray 2BL).

In some embodiments, a dye further comprises a pharmaceutically acceptable salt.

Non-limiting examples of pharmaceutical excipients include but are not limited to: fillers, plasticizers, flavors, preservatives, and sorbents.

In some embodiments, the additive has adhesiveness to the surface of the adapter. In some embodiments, the additive forms van der Waals bonds with the wall. In some embodiments, the additive forms p-p stacking interactions with the wall. In some embodiments, the additive is absorbed to the wall.

In some embodiments, the additive has at least one functional group selected from: an aromatic ring, an alkyl chain, and a charged residue. In some embodiments, the additive is a neutral compound comprising a hydrogen donor or acceptor. In some embodiments, the additive comprises an aromatic ring.

In some embodiments, the additive is a charged compound. In some embodiments, the additive is an amphiphilic substance containing a hydrophobic segment (e.g. an aromatic ring) and a hydrophilic segment (e.g. a charged group).

In some embodiments, the additive is absorbed to the adhesive. In some embodiments, the additive is bound to the adhesive via non-covalent bonds. In some embodiments, the additive is incorporated within the adhesive. In some embodiments, the additive is incorporated within the coating layer. In some embodiments, the additive is bound to the outer surface of the coating layer.

In some embodiments, the additive is a dye. In some embodiments, the additive is selected from an anionic and a cationic dye.

### Manufacturing Process (Coating)

In some embodiments, provided herein a method of coating an adapter. In some embodiments, the method comprises mixing an additive with a solvent, thereby obtaining a coating solution comprising the additive. In some embodiments, the coating solution further comprises an adhesive. In some embodiments, the method comprises mixing the additive and the adhesive with a solvent, thereby obtaining a coating solution comprising the additive and the adhesive. In some embodiments, the coating solution further comprises a constituent. In some embodiments, the constituent is selected from the group consisting of: a surfactant, a tackifier, a crosslinking agent, a wetting agent, and a binder or any combination thereof.

The constituent mentioned herein may be used in the amounts suitable for such purposes, for example the wetting agent and the emulsifier in a concentration of 0.5 to 30 w/w %, the thickeners in a concentration of 0.1 to 30 w/w %.

Non-limiting examples of solvents that can be used to prepare the coating solution include those that will dissolve solid components of the coating, that are compatible with the coating and the adapter, and can ensure uniform coverage of the surface of the adapter. Potential solvents include, but are not limited to, the following: water, ethanol, methanol, ethylene glycol, or any combination thereof.

In some embodiments, the coating solution is an aqueous solution. In some embodiments, depending on the coating composition, the solution is a buffered solution with an acidic pH (e.g. 0-6). In some embodiments, the solution has a basic pH (e.g. 8-14). In some embodiments, the solution has a pH in the range of 6 to 8. In some embodiments, the coating solution is an aqueous alcoholic solution. In some embodiments, the coating solution is a dispersion. In some embodiments, the coating solution is an emulsion.

In some embodiments, the method comprises mixing an additive and an adhesive at a w/w (weight/weight) ratio ranging from 1:1 to 1:50 with a solvent, thereby obtaining a coating solution comprising the additive and the adhesive.

In some embodiments, the coating is applied to the adapter by a method selected from: spin coating, spray coating, spray and spin coating, curtain coating, flow coating, dip coating, injection molding, casting, roll coating, wire coating, chemical vapor deposition, physical vapor deposition and any of the methods used in preparing overlays. Generally, the application method selected will depend upon, among other things, chemical properties of materials composing the coating, the thickness of the desired coating, the geometry of the surface to which the coating is applied, and the viscosity of the coating.

In some embodiments, the method optionally comprises cleaning at least a portion of the adapter surface prior to applying any coatings thereto. This can be done for the purposes of cleaning and/or promoting adhesion of the coating. Effective treatment techniques are known to those skilled in the art.

In some embodiments, the coating is applied to the adapter by dip coating. In some embodiments, the method comprises contacting the adapter with the coating solution. In some embodiments, the method comprises contacting at least a part of the adapter with the coating solution. In some embodiments, the adapter is placed into a container containing the coating solution.

In some embodiments, the method comprises:

(i) providing the coating to conditions suitable for melting, thereby obtaining a molten coating;

(ii) contacting the adapter with the molten coating;

(iii) optionally, curing the molten coating, to obtain a coated adapter.

In some embodiments, the method further comprises applying a vacuum after contacting the coating solution or the molten coating with the adapter, to remove air from the coating. In some embodiments, air removal is performed in order to obtain a uniform coating. In some embodiments, air removal is performed in order to facilitate penetration of the coating solution into the smallest parts of the adaptor.

In some embodiments, the method comprises drying the coating solution at 10 to 90° C., thereby obtaining a coated adapter. In some embodiments, drying is performed by convection drying, such as by applying a hot gas stream to a coated surface. In some embodiments, drying is performed by cold drying, such as by applying a de-humidified gas stream to the surface. In some embodiments, drying is performed by infrared (IR) drying. In some embodiments, drying is performed by microwave drying. In some embodiments, the method further comprises reacting one or more components of the coating (e.g. by photo-crosslinking). Generally, the drying method and exact drying conditions selected will depend upon, among other things, chemical and physical properties of materials composing the coating, and chemical and physical properties of the adaptor (e.g. thermal stability).

In some embodiments, the method further comprises vacuum drying of the coating solution. In some embodiments, the method comprises a partial drying of the coating.

In some embodiments, a container comprising the coating solution and the adapter is provided to conditions suitable for drying, such as a temperature ranging from 10 to 90° C. In some embodiments, conditions suitable for drying further comprise applying a vacuum.

In some embodiments (e.g., wherein the adapter has more than one opening), one opening of the adapter is closed by a plug to obtain a chamber for holding the coating solution. In some embodiments, the coating solution is provided via a second opening to the chamber, thereby contacting the adapter with the coating solution.

In some embodiments, the adapter holding the coating solution, is provided to conditions suitable for drying as described herein.

In some embodiments (e.g. when the coating comprises multiple layers), the method further comprises applying a second layer. In some embodiments, an intermediate layer is applied to attach the first layer to the second layer.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. An adapter, comprising:
at least one wall defining a chamber;
at least one opening within said wall; and
a water-soluble additive coating at least a portion of said wall such that fluid flowing from one opening through said chamber dissolves said additive within said chamber.

2. The adapter of claim 1, wherein said water-soluble additive comprises an adhesiveness property in relation to the wall.

3. The adapter of claim 1, wherein said water-soluble additive dissolves homogenously within the fluid.

4. The adapter of claim 1, wherein said water-soluble additive is inert to the fluid.

5. The adapter of claim 1, wherein said water-soluble additive is selected from the group consisting of: a dye, a pharmaceutical, and a pharmaceutical excipient or any combination thereof.

6. (canceled)

7. The adapter of claim 1, wherein said coating further comprises a water-soluble adhesive.

8. The adapter of claim 7, wherein said coating comprises the water-soluble additive and the water-soluble adhesive at a w/w (weight/weight) ratio of 1:1 to 1:50.

9. The adapter of claim 7, wherein said adhesive is selected from the group consisting of: a water-soluble saccharide, a peptide, a protein, a low-molecular weight surfactant, and a water-soluble adhesive polymer.

10. The adapter of claim 9, wherein said adhesive is selected from (i) an adhesive polymer selected from: a poly-phosphazene, a poly-phosphoester, a poly-oxazoline, a poly-[divinylether-maleic anhydride], a poly-[N(2-hydroxypropyl)methacrylamide], a poly-acrylamide, a poly-acrylic acid, a poly-vinyl alcohol, a poly-vinyl pyrrolidone, a poly-ethylene glycol or any combination thereof; (ii) a water-soluble saccharide selected from a monosaccharide, an oligosaccharide, and a water-soluble polysaccharide or any combination thereof.

11. (canceled)

12. (canceled)

13. The adapter of claim 1, wherein said opening is configured to couple to at least one of a vial, a syringe, a compartment, a pipe, a funnel, a beaker, a tube, a pipet, and an adapter.

14. The adapter of claim 1, wherein said chamber comprises at least two openings and at least one channel between said opening.

15. The adapter of claim 14, wherein at least a portion of said at least one channel is coated by said water-soluble additive.

16. The adapter of claim 1, wherein said chamber comprises at least two openings and wherein the path of the channel between said openings is longer than the minimal distance between said openings.

17. The adapter of claim 1, wherein at least a portion of the surface of said wall within said chamber or the surface of said channel is irregular, textured, and/or comprises irregularities.

18. (canceled)

19. The adapter of claim 17, wherein said irregular surface, textured surface, and/or irregularities comprise at least one of a nanostructure, microstructure, or any combination thereof.

20. The adapter of claim 17, wherein said water-soluble additive covers at least a portion of the irregular surface, textured surface, and/or irregularities.

21. The adapter of claim 1, wherein the surface area of at least a portion of the inner surface of the wall inner surface is larger than the surface area of the corresponding outer surface of the portion of the wall in relation to the chamber.

22. A kit for mixing an additive with fluid, comprising:
at least one of fluid source and/or a fluid drain; and
at least one adapter configured to couple to said fluid source and/or said fluid drain, comprising
at least one wall defining a chamber;
at least one opening within said wall configured to couple to said fluid source and/or fluid drain; and
a water-soluble additive coating at least a portion of said wall such that fluid flowing within said chamber dissolves said additive.

23. The kit according to claim 22, wherein said fluid source and/or fluid drain is at least one of a vial, a syringe, a compartment, a pipe, a funnel, a beaker, a tube, a pipet, and an adapter.

24. A method for mixing water-soluble additive with fluid, comprising:
streaming fluid into an adapter through at least one opening;
streaming fluid through a chamber of said adapter such that water-soluble additive coating at least a portion of the wall of said chamber dissolves said fluid; and
ejecting a homogenous mixture of said fluid and said water-soluble additive through at least one opening of said adapter.

25. (canceled)

26. (canceled)

27. (canceled)
